SEQUENCE LISTING (14 sequences)

SEQ ID NO: 1 - DNA, 1050 bp, Leuconostoc mesenteroides
  source 1..1050: mol_type = genomic DNA
cgccttggtcctgcctataacggctccttcctaaaaggtataggccaccggctttgggcattacaaactcccatggtgtgacgggcggtgtgtacaagacccgggaacgtattcaccgcggcgtgctgatccgcgattactagcgattccgacttcatgtagtcgagttgcagactacaatccgaactgagacgtactttaagagattagctcaccctcgcgggttggcaactcgttgtatacgccattgtagcacgtgtgtagcccaggtcataaggggcatgatgatctgacgtcgtccccgccttcctccggtttgtcaccggcagtctcgctagagtgcccatctgaatgctggcaactaacaataagggttgcgctcgttgcgggacttaacccaacatctcacgacacgagctgacgacgaccatgcaccacctgtcactttgtctccgaagagaacacttctatctctaaaagcttcaaaggatgtcaagacctggtaaggttcttcgcgttgcttcgaattaaaccacatgctccaccgcttgtgcgggtccccgtcaattcctttgagtttcaaccttgcggtcgtactccccaggcggaacacttaatgcgttagcttcggcactaagaggcggaaacctcctaacacctagtgttcatcgtttacggtgtggactaccagggtatctaatcctgtttgctacccacactttcgagcctcaacgtcagttgcagtccagtaagccgccttcgccactggtgttcttccatatatctacgcattccaccgctacacatggagttccacttacctctactgcactcaagttaaccagtttccaatgccattccggagttgagctccgggctttcacatcagacttaataaaccgtctgcgctcgctttacgcccaataaatccggataacgctcgggacatacgtattaccgcggctgctggcacgtatttagccgtccctttctggtatggtaccgtcaaactaaatcatttcctatctagctgtctcccatacacagtgc

SEQ ID NO: 2 - DNA, 1047 bp, Leuconostoc sp.
  source 1..1047: mol_type = genomic DNA
atgaaggctttcgggtcgtaaagcactgttgtatgggaagaacagctagaatagggaatgattttagtgtgacggtaccataccagaaagggacggctaaatacgtgccagcagccgcggtaatacgtatgtcccgagcgttatccggatttattgggcgtaaagcgagcgcagacggttgtttaagtctgatgtgaaagcccggagctcaactccggaaaggcattggaaactggacaacttgagtgcagtagaggtaagtggaactccatgtgtagcggtggaatgcgtagatatatggaagaacaccagcggcgaaggcggcttactggactgtaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagatgggcactctagcgagactgccggtgacaaaccggaggaaggcggggacgacgtcagatcatcatgccccttatgacctgggctacacacgtgctacaatggcgtatacaacgagttgccaacctgcgaaggtgagctaatctcttaaagtacgtctcagttcggactgcagtctgcaactcgactgcacgaagtcggaatcgctagtaatcgcggatcagcacgccgcggtgaatacgttcccgggtcttgtacacaccgcccgtcacaccatgggagtttgtaatgcccaaagccggtggcctaaccgtaagga

SEQ ID NO: 3 - DNA, 1029 bp, Leuconostoc lactis
  source 1..1029: mol_type = genomic DNA
cagaaagggacggctaaatacgtgccagcagccgcggtaatacgtatgtcccgagcgttatccggatttattgggcgtaaagcgagcgcagacggttgattaagtctgatgtgaaagcccggagctcaactccggaatggcattggaaactggttaacttgagtgttgtagaggtaagtggaactccatgtgtagcggtggaatgcgtagatatatggaagaacaccagtggcgaaggcggcttactggacaacaactgacgttgaggctcgaaagtgtgggtagcaaacaggattagataccctggtagtccacaccgtaaacgatgaatactaggtgttaggaggtttccgcctcttagtgccgaagctaacgcattaagtattccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagttgggcactctagcgagactgccggtgacaaaccggaggaaggcggggacgacgtcagatcatcatgccccttatgacctgggctacacacgtgctacaatggcgtatacaacgagttgccaacccgcgagggtgagctaatctcttaaagtacgtctcagttcggactgcagtctgcaactcgactgcacgaagtcggaatcgctagtaatcgcggatcagcacgccgcggtgaatacgttcccgggtcttgtacacaccgcccgtcacaccatgggagtttgtaatgcccaaagccggtggcctaaccttcgggagggagccgtctaaggcaggacagatgactagggtgaagtcgtaacaaggtagccgtaggagaacc

SEQ ID NO: 4 - DNA, 984 bp, Leuconostoc citrcum
  source 1..984: mol_type = genomic DNA
acgtgccagcagccgcggtaatacgtatgtcccgagcgttatccggatttattgggcgtaaagcgagcgcagacggttgattaagtctgatgtgaaagcccggagctcaactccggaatggcattggaaactggttaacttgagtgttgtagaggtaagtggaactccatgtgtagcggtggaatgcgtagatatatggaagaacaccagtggcgaaggcggcttactggacaacaactgacgttgaggctcgaaagtgtgggtagcaaacaggattagataccctggtagtccacaccgtaaacgatgaatactaggtgttaggaggtttccgcctcttagtgccgaagctaacgcattaagtattccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagttgggcactctagcgagactgccggtgacaaaccggaggaaggcggggacgacgtcagatcatcatgccccttatgacctgggctacacacgtgctacaatggcgtatacaacgagttgccaacctgcgaaggtgagctaatctcttaaagtacgtctcagttcggactgcagtctgcaactcgactgcacgaagtcggaatcgctagtaatcgcggatcagcacgccgcggtgaatacgttcccgggtcttgtacacaccgcccgtcacaccatgggagtttgtaatgcccaaagccggtggcctaaccttcgggagggagccgtctaaggcaggacagatgactggggcgaagt

SEQ ID NO: 5 - DNA, 1032 bp, Leuconostoc carnosum
  source 1..1032: mol_type = genomic DNA
agaaaagttaaggtagggaatgaccttagcctgacggtaccataccagaaagggacggctaaatacgtgccagcagccgcggtaatacgtatgtcccgagcgttatccggatttattgggcgtaaagcgagcgcagacggttggttaagtctgatgtgaaagcccggagctcaactccggaaaggcattggaaactggttaacttgagtgcagtagaggtaagtggaactccatgtgtagcggtggaatgcgtagatatatggaagaacaccagcggcgaaggcggcttactggactgtaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagaaatagaagtgttctcttcggagacaaagtgacaggtggtgcatggtcgtcgtcagctcgtgtcgtgagatgttgggttaagtcccgcaacgagcgcaacccttattgttagttgccagcattcagatgggcactctagcgagactgccggtgacaaaccggaggaaggcggggacgacgtcagatcatcatgccccttatgacctgggctacacacgtgctacaatggcgtatacaacgagttgccaacctgcgaaggtgagctaatctcttaaagtacgtctcagttcggactgcagtctgcaactcgactgcacgaagtcggaatcgctagtaatcgcggatcagcacgccgcggtgaatacgttcccgggtcttgtacacaccgcccgtcacaccatggaagtttgtaatgcccaaagccggtggcctaaccgtaaggagggaaccgtctaaggcaggacag

SEQ ID NO: 6 - DNA, 1052 bp, Leuconostoc mesenteroides
  source 1..1052: mol_type = genomic DNA; sub_species = subsp.
gatgaaggctttcgggtcgtaaagcactgttgtatgggaagaacagctagaataggaaatgattttagtttgacggtaccataccagaaagggacggctaaatacgtgccagcagccgcggtaatacgtatgtcccgagcgttatccggatttattgggcgtaaagcgagcgcagacggtttattaagtctgatgtgaaagcccggagctcaacttcggaatggcattggaaactggttaacttgagtgcagtagaggtaagtggaactccatgtgtagcggtggaatgcgtagatatatggaagaacaccagtggcgaaggcggcttactggactgcaactgacgttgaggctcgaaagtgtgggtagcaaacaggattagataccctggtagtccacaccgtaaacgatgaacactaggtgttaggaggtttccgcctcttagtgccgaagctaacgcattaagtgttccgcctggggagtacgaccgcaaggttgaaactcaaaggaattgacggggacccgcacaagcggtggagcatgtggtttaattcgaagcaacgcgaagaaccttaccaggtcttgacatcctttgaagcttttagagatagaagtgttctcttcggagacaaagtgacaggtggtgcatggtcgtcgtcagctcgtgtcgtgagatgttgggttaagtcccgcaacgagcgcaacccttattgttagttgccagcattcagatgggcactctagcgagactgccggtgacaaaccggaggaaggcggggacgacgtcagatcatcatgccccttatgacctgggctacacacgtgctacaatggcgtatacaacgagttgccaacccgcgagggtgagctaatctcttaaagtacgtctcagttcggattgtagtctgcaactcgactacatgaagtcggaatcgctagtaatcgcggatcagcacgccgcggtgaatacgttcccgggtcttgtacacaccgcccgtcacaccatgggagtttgtaatgcccaaagccggtggcctaaccttttaggaagg

SEQ ID NO: 7 - DNA, 1045 bp, Leuconostoc pseudomesenteroides
  source 1..1045: mol_type = genomic DNA
gtggacaacctgcctcaaggctggggataacatttggaaacagatgctaataccgaatgaaacttagtgtcgcatgacacgaagttaaaaggcgctttggcgtcacctagagatggatccgcggtgcattagttagttggtggggggcagccctaccaagacaatgatgcatagctgagttgagagactgatcggccacattgccgactgaaaccatcaaaaaaactttatgggggagggtgcagttccgaaatctgccaaaatcggcgaaagcctgatggagcagcgccgcgtgtgttttggagcgctttcggctcgtatagcactgttgaatgctaagaacagctagaatagttgaatgattttagtttgacggaaccataccagaaaggaacggctaactacgcgcgagcagccgcggtaaccacgtatgtcccgagcgttattcgggatttattgagcgtaaagcgagcgcttacgtttgattaagtctgatgtgaaagctggagctcggctccggaatggcattgaaaactggttaacttgagtgcagtagaggtcagtggaactccgtgtgtagcgatgtaatgcgaagatatatgggaggaacaccagtgtcgaggcggcttactggattgtaacttgaagttgaggctcgcagtgtgattgctacccggagtagataccctggtagtccacaccgtaaacgatgaagactattgtgtctatgcaggtgtgcccgacctctaacgctagaagctcaccgccattgaggtgttcccgcccccgggggagtaccactcgcaaagatttgatagtgcaagcgcgaactagacggggacccggacgagggctgatgtagcatggagcctaaatccgaagccacgcgcgttaggactcttacctaggtcttgacattcagttgagaaggcttctctcagataaaagtgtacctctcggggacgaggtgactgggcgtcgcactgggccgatcgtcagtctaggttccggacatgtgtgccatcagactcgtaacgagggctaa

SEQ ID NO: 8 - DNA, 1042 bp, Leuconostoc fallax
  source 1..1042: mol_type = genomic DNA
ggaaatgatgttaatgtgacggtaccataccagaaagggacggctaaatacgtgccagcagccgcggtaatacgtatgtcccgagcgttatccggatttattgggcgtaaagcgagcgcagacggttcgttaagtctgaagtgaaagcccacagctcaactgtggaatggctttggaaactggcgaacttgagtgcagtagaggcaagtggaactccatgtgtagcggtggaatgcgtagatatatggaagaacaccagtggcgaaggcggcttgctggactgtaactgacgttgaggctcgaaagtgtgggtagcaaacaggattagataccctggtagtccacaccgtaaacgatgagtgctaggtgttaggaggtttccgcctcttagtgccggagctaacgcattaagcactccgcctggggagtacgaccgcaaggttgaaactcaaaggaattgacggggacccgcacaagcggtggagcatgtggtttaattcgaagcaacgcgaagaaccttaccaggtcttgacatcctttgaagatgttagagataatattgttctcttcggagacaaagtgacaggtggtgcatggtcgtcgtcagctcgtgtcgtgagatgttgggttaagtcccgcaacgagcgcaacccttatgtttagttgccagcatttagttgggcactctagacagactgccggtgacaaaccggaggaaggcggggatgacgtcagatcatcatgccccttatgacctgggctacacacgtgctacaatggcgtatacaacgagttgcgaaccggcgacggtaagctaatctcttaaagtacgtctcagttcggattgtagtctgcaactcgactacatgaagtcggaatcgctagtaatcgcggatcagcatgccgcggtgaatacgttcccgggtcttgtacacaccgcccgtcacaccatgagagtttgtaacacccaaagccggtgggctaaccttcgggaggtagccgtctaaggtgggacagatgattagggtgaagtcgtaacaag

SEQ ID NO: 9 - DNA, 21 bp, Synthetic construct
  source 1..21: mol_type = other DNA
ggccaagtcaagtatgggttg

SEQ ID NO: 10 - DNA, 22 bp, Synthetic construct
  source 1..22: mol_type = genomic DNA
acagacacgttctgaatggaga

SEQ ID NO: 11 - DNA, 21 bp, Synthetic construct
  source 1..21: mol_type = genomic DNA
actccgactctgggctcttca

SEQ ID NO: 12 - DNA, 18 bp, Synthetic construct
  source 1..18: mol_type = genomic DNA
ggcctccgggctgtagaa

SEQ ID NO: 13 - DNA, 22 bp, Synthetic construct
  source 1..22: mol_type = genomic DNA
ttcactccatctcaagcatcga

SEQ ID NO: 14 - DNA, 23 bp, Synthetic construct
  source 1..23: mol_type = genomic DNA
ggacagtaggttttcagccaaca

(19) United States
(12) Patent Application Publication
(10) Pub. No.: US 2025/0325604 A1
(43) Pub. Date: Oct. 23, 2025

(54) METHOD FOR REDUCING BODY FAT, REDUCING APPETITE, PROMOTING KETONE PRODUCTION, OR REGULATING FASTING BLOOD GLUCOSE BY USING LEUCONOSTOC MESENTEROIDES AND/OR SUPERNTANT THEREOF

(71) Applicant: TCI CO., LTD., Taipei (TW)

(72) Inventors: YUNG-HSIANG LIN, Taipei (TW); DI CHANG, Taipei (TW)

(21) Appl. No.: 19/064,723

(22) Filed: Feb. 27, 2025

Related U.S. Application Data
(60) Provisional application No. 63/636,114, filed on Apr. 19, 2024.

(51) Int. Cl. A61K 35/744; A61K 35/00; A61P 3/04; A61P 3/06; A61P 3/08; A61P 5/50; C12N 1/20; C12R 1/01

(52) U.S. Cl. CPC A61K 35/744; A61P 3/04; A61P 3/06; A61P 3/08; A61P 5/50; C12N 1/205; A61K 2035/115; C12R 2001/01

(57) ABSTRACT

Use of Leuconostoc mesenteroides in preparing compositions for reducing body fat, decreasing appetite, promoting ketone production or regulating fasting blood glucose is provided, where the Leuconostoc mesenteroides is Leuconostoc mesenteroides subsp. and the Leuconostoc mesenteroides was deposited at Food Industry Research and Development Institute under the accession number BCRC911146 or German Collection of Microorganisms and Cell Cultures under the accession number DSM34443.

### CROSS-REFERENCE TO RELATED APPLICATION

This application claims the benefit of U.S. provisional application Ser. No. 63/636,114, filed on Apr. 19, 2024. The entirety of the above-mentioned patent application is hereby incorporated by reference herein and made a part of the specification.

### REFERENCE OF AN ELECTRONIC SEQUENCE LISTING

The contents of the electronic sequence listing (P245885USI_20250610.xml; Size: 22,033 bytes; and Date of Creation: Jun. 10, 2025) is herein incorporated by reference in its entirety.

### BACKGROUND

### Technical Field

The present invention relates to Leuconostoc mesenteroides, and particularly relates to use of Leuconostoc mesenteroides subsp. Mesenteroides and/or supernatant thereof in preparing compositions for reducing body fat, reducing appetite, promoting ketone production, or regulating fasting blood glucose.

### Related Art

Leuconostoc mesenteroides is an important strain of Leuconostoc of lactic acid bacteria, which grows well in anaerobic culture and optimally grows at the temperature of 30° C. to 40° C. Leuconostoc mesenteroides has an antagonistic effect on common pathogenic bacteria such as Shigella castellani, salmonella, and Staphylococcus aureus, and it is a probiotic with development potential.

### SUMMARY

In view of above, the present invention provides Leuconostoc mesenteroides and/or supernatant thereof, and the Leuconostoc mesenteroides is Leuconostoc mesenteroides subsp. Mesenteroides under accession number BCRC911146 or DSM34443.

In some embodiments, a method for reducing body fat includes: administering to a subject in need thereof an effective dose of Leuconostoc mesenteroides and/or supernatant thereof.

In some embodiments, the Leuconostoc mesenteroides can promote fat metabolism.

In some embodiments, the Leuconostoc mesenteroides can reduce lipid droplet accumulation of adipocytes.

In some embodiments, the Leuconostoc mesenteroides can promote cholesterol metabolism.

In some embodiments, the Leuconostoc mesenteroides can increase the content of high-density cholesterol in blood.

In some embodiments, the Leuconostoc mesenteroides can promote the production of high-density cholesterol. In some embodiments, the Leuconostoc mesenteroides can promote the increase of expression level of CETP gene, SCARB1 gene or LDLR gene.

In some embodiments, a method for reducing appetite includes: administering to a subject in need thereof an effective dose of Leuconostoc mesenteroides and/or supernatant thereof.

In some embodiments, the Leuconostoc mesenteroides can promote the secretion of incretin.

In some embodiments, the Leuconostoc mesenteroides can promote the secretion of GLP-1 by intestinal cells.

In some embodiments, a method for promoting ketone production includes: administering to a subject in need thereof an effective dose of Leuconostoc mesenteroides or supernatant thereof.

In some embodiments, the Leuconostoc mesenteroides can produce ketone.

In some embodiments, the Leuconostoc mesenteroides is used for producing β-hydroxybutyrate.

In some embodiments, the Leuconostoc mesenteroides can increase the concentration of ketone in serum.

In some embodiments, the Leuconostoc mesenteroides can increase the content of ketone in urine.

In some embodiment, provided is use of Leuconostoc mesenteroides and/or supernatant thereof in preparing a composition for regulating fasting blood glucose.

In some embodiments, the Leuconostoc mesenteroides can reduce an insulin resistance index.

In some embodiments, the Leuconostoc mesenteroides can promote the secretion of incretin.

In some embodiments, the Leuconostoc mesenteroides can promote the secretion of GLP-1 by intestinal cells.

In some embodiments, the Leuconostoc mesenteroides can reduce the concentration of glycated albumin.

In conclusion, the Leuconostoc mesenteroides and/or supernatant thereof in any embodiment above can promote fat metabolism, reduce lipid droplet accumulation of adipocytes, promote cholesterol metabolism, increase the content of high-density cholesterol in blood, promote production of high-density cholesterol and promote the increase of expression level of CETP gene, SCARB1 gene or LDLR gene, thus achieving the use of reducing body fat. The Leuconostoc mesenteroides and/or supernatant thereof in any embodiment can promote secretion of incretin and/or promote secretion of GLP-1 by intestinal cells, thus achieving the use of reducing appetite. The Leuconostoc mesenteroides and/or supernatant thereof in any embodiment can produce ketone, β-hydroxybutyrate, increase the concentration of ketone in serum and increase the content of ketone in urine, thus achieving the use of promoting ketone production. The Leuconostoc mesenteroides and/or supernatant thereof in any embodiment can reduce the insulin resistance index, promote secretion of incretin and/or promote secretion of GLP-1 by intestinal cells and reduce the concentration of glycated albumin, thus achieving the use of regulating fasting blood glucose.

### BRIEF DESCRIPTION OF THE DRAWINGS

FIG. 1 shows a phylogenetic tree of Leuconostoc mesenteroides TCI818.

FIG. 2 shows a cell state in a fat accumulation inhibition test of Leuconostoc mesenteroides.

FIG. 3 shows a result of a fat accumulation inhibition test of Leuconostoc mesenteroides.

FIG. 4 shows a result of a liver cholesterol metabolism promotion test of Leuconostoc mesenteroides.

FIG. 5 shows a result of a cholesterol-related gene expression test of Leuconostoc mesenteroides.

FIG. 6 shows a result of an incretin promotion test of Leuconostoc mesenteroides.

FIG. 7 shows a result of a BHB secretion promotion test of Leuconostoc mesenteroides.

FIG. 8 shows a result of concentration change of ketone in serum in human experiments.

FIG. 9 shows a result of concentration change of ketone in urine in human experiments.

FIG. 10 shows a result of content change of high-density cholesterol in human experiments.

FIG. 11 shows a result of average body fat rate change in human experiments.

FIG. 12 shows a result of average trunk fat weight change in human experiments.

FIG. 13 shows a result of average visceral fat area change in human experiments.

FIG. 14 shows a result of average skeletal muscle weight change in human experiments.

FIG. 15 shows a result of average basal metabolic rate change in human experiments.

### DETAILED DESCRIPTION

The present invention provides Leuconostoc mesenteroides, which is a strain isolated from Allium tuberosum Rottler ex Spreng. The Leuconostoc mesenteroides is deposited at Food Industry Research and Development Institute under the accession number BCRC911146 or German Collection of Microorganisms and Cell Cultures under the accession number DSM34443.

The Leuconostoc mesenteroides of the present invention may also be referred to as Leuconostoc mesenteroides TCI818, it is a gram-positive bacterium of Leuconostoc, and belongs to facultative anaerobes, and is Leuconostoc mesenteroides belonging to Leuconostoc mesenteroides subsp. In some embodiments, supernatant is a clear liquid that remains above the pellet after centrifugation, precipitation, or filtration of a culture broth of Leuconostoc mesenteroides. In some embodiments, metabolites are the intermediate products produced during metabolism, and catalyzed by various enzymes that occur naturally within the Leuconostoc mesenteroides cells. Herein, the supernatant contains the metabolites of Leuconostoc mesenteroides.

In some embodiments, provided is use of Leuconostoc mesenteroides or supernatant thereof in preparing compositions for increasing muscle and reducing fat; the Leuconostoc mesenteroides is Leuconostoc mesenteroides subsp. Mesenteroides deposited at Food Industry Research and Development Institute under accession number BCRC911146 or Leuconostoc mesenteroides subsp. Mesenteroides deposited at German Collection of Microorganisms and Cell Cultures (DSMZ, Address: Inhoffenstr. 7 B D-38124 Braunschweig), Germany, in accordance with the Budapest Treaty.) under accession number DSM34443.

In some embodiments, provided is use of Leuconostoc mesenteroides and/or metabolites thereof in preparing a composition for reducing body fat.

In some embodiments, provided is use of Leuconostoc mesenteroides and/or supernatant thereof in preparing a composition for reducing appetite.

In some embodiments, provided is use of Leuconostoc mesenteroides and/or supernatant thereof in preparing a composition for promoting ketone production.

In some embodiments, provided is use of Leuconostoc mesenteroides and/or supernatant thereof in preparing a composition for regulating fasting blood glucose.

In some embodiments, the Leuconostoc mesenteroides and/or supernatant thereof can promote fat metabolism.

In some embodiments, the Leuconostoc mesenteroides and/or supernatant thereof can reduce lipid droplet accumulation of adipocytes.

In some embodiments, the Leuconostoc mesenteroides and/or supernatant thereof can promote cholesterol metabolism.

In some embodiments, the Leuconostoc mesenteroides and/or supernatant thereof can increase the content of high-density cholesterol in blood.

In some embodiments, the Leuconostoc mesenteroides can promote the production of high-density cholesterol. In some embodiments, the Leuconostoc mesenteroides can promote the increase of expression level of CETP gene, SCARB1 gene or LDLR gene.

The protein encoded by the CETP gene (Gene ID: 1071) is a plasma cholesterol ester transfer protein, which mainly involved in the transport of cholesterol ester and triglyceride in plasma. The protein is helpful for regulating cholesterol level, and when CETP expression rises, the cholesterol ester in HDL can be more effectively transferred into LDL or VLDL. In a case of synchronous up-regulation of a low-density lipoprotein receptor (LDLR), the cholesterol transferred into the LDL/VLDL is easier to be removed by the liver. Overall reverse cholesterol transport (namely transport of cholesterol from the periphery to the liver) is promoted, and finally the cholesterol burden in blood and cardiovascular risk are reduced.

The SCARB1 gene (Gene ID: 949) encodes a scavenger receptor BI (SR-BI), which is mainly responsible for the internalization and transport of high-density lipoprotein (HDL) cholesterol. This protein helps cholesterol to transfer from plasma to the liver. It is a receptor for high-density protein and regulates the excretion of the high-density protein. The enhanced SR-BI function means that reverse cholesterol transport can be more efficient, which is conductive to the clearing of excessive cholesterol in the periphery, and the reduction in risk of atherosclerosis.

The LDLR gene (Gene ID: 3949) encodes a low-density lipoprotein receptor. It is mainly used for identifying and clearing low-density lipoprotein (LDL) cholesterol in blood, and LDL is internalized into the liver to help regulate blood lipid level. When LDLR expression rises, the uptake capacity of liver cells to LDL is enhanced, and accordingly the concentration of LDL in blood is reduced. Excessive LDL cholesterol in blood is cleared to help reduce the accumulation of cholesterol in artery walls and reduce the risk of cardiovascular diseases. Moreover, when CETP transfers cholesterol from HDL to LDL, the up-regulated LDLR can clear the LDL more effectively, thereby further improving the overall cholesterol balance.

In some embodiments, the Leuconostoc mesenteroides can promote the secretion of incretin. Common incretin include Glucose-dependent insulinotropic polypetide (GIP) and Glucagon-like peptide-1 (GLP-1). The GLP-1 can inhibit the secretion of the glucagon, the GIP increases the secretion of the glucagon, and both act in a glucose-dependent manner. The GIP directly acts on adipose tissues to promote energy storage, and enhances bone formation by stimulating osteoblast proliferation and inhibiting cell apoptosis. On the contrary, the GLP-1 plays a role in regulating glucose by slowing gastric emptying and inhibiting secretion of glucose-dependent glucagon. Moreover, the GLP-1 also has the application of promoting fat metabolism and transforming white fat into brown fat.

In some embodiments, the Leuconostoc mesenteroides can promote the secretion of GLP-1 by intestinal cells.

In some embodiments, the Leuconostoc mesenteroides can produce ketone. Ketogenesis is a metabolic response triggered when the concentration of glucose in blood decreases or carbohydrate reserves (such as liver sugar) in cells are used up. In this process, energy in fatty acid is transformed into ketone to provide an alternative energy source.

In some embodiments, the Leuconostoc mesenteroides is used for producing β-hydroxybutyrate. The β-hydroxybutyrate (BHB) is one of ketone, and it is usually synthesized in the liver. Here, the BHB can directly act on L cells in the intestinal tract to promote the L cells to secrete more GLP-1, and the BHB can be combined with receptors on the surfaces of the L cells to activate signal paths in the cells so as to promote synthesis and secretion of the GLP-1. The BHB can also indirectly promote the secretion of the GLP-1 by changing the composition of intestinal microflora. That is, the change in intestinal microflora will influence the intestinal environment and the functions of the L cells, thus influencing the secretion of the GLP-1. The BHB can activate specific metabolic induction molecules such as AMPK (AMP-activated protein kinase), and the molecules play an important role in energy metabolism and nutrition induction, and finally influence the secretion of the GLP-1.

In some embodiments, the Leuconostoc mesenteroides can increase the concentration of ketone in serum. In some embodiments, the Leuconostoc mesenteroides can increase the content of ketone in urine. The ketone can promote the intestinal tract to secrete the GLP-1.

In some embodiments, the Leuconostoc mesenteroides can reduce an insulin resistance index.

In some embodiments, the Leuconostoc mesenteroides can reduce the concentration of glycated albumin. The glycated albumin has certain importance on blood glucose monitoring for diabetic patients. This test can be performed without fasting, and because the half-life period of the albumin is about 2-4 weeks, it can reflect the short-term blood glucose control situation.

In some embodiments, the effective dose of the Leuconostoc mesenteroides is 100 mg/d.

In some embodiments, the compositions above include Leuconostoc mesenteroides or supernatant thereof in a specific content.

In some embodiments, the compositions may be non-medical health products, food products or food additives. In other words, the health products, the food products or the food additives contain Leuconostoc mesenteroides in a specific dosage.

In some embodiments, the health products, the food products or the food additives above may further contain a food industry acceptable carrier that is widely used. For example, the food industry acceptable carrier may contain one or more of the following agents: a solvent, a buffer, an emulsifier, a suspending agent, a decomposer, a disintegrating agent, a dispersing agent, a binder, an excipient, a stabilizing agent, a chelating agent, a diluent, a gelling agent, a preservative, a wetting agent, a lubricant, an absorption delaying agent, liposome and the like. The selection and quantity of these agents fall within the expertise and routine technical scope of those familiar with the technology.

In some embodiments, the acceptable carriers of the health products, the food products or the food additives above contain solvents selected from the group consisting of water, normal saline, phosphate buffered saline (PBS) and an aqueous solution containing alcohol.

In some embodiments, the food products may be but not limited to beverages, fermented foods, bakery products, non-medical healthy foods and dietary supplements.

In some embodiments, the composition contains the Leuconostoc mesenteroides in a specific dosage. This means that the effective dosage of Leuconostoc mesenteroides TCI818 is 100 mg/d. Specifically, assuming that one unit of the composition is administered daily and the Leuconostoc mesenteroides is dry powder, the composition contains at least 100 mg of Leuconostoc mesenteroides. In some embodiments, the subject is human. In some embodiments, the composition may only contain the Leuconostoc mesenteroides. In some other embodiments, the composition contains the Leuconostoc mesenteroides and at least one food additive, and the food additive is at least one selected from the group consisting of skim milk powder, trehalose and lactose. In some embodiments, the composition consists of the Leuconostoc mesenteroides, skim milk powder, trehalose and lactose. In some examples, 2.2% (w/w) trehalose, 4.6% (w/w) lactose and 10% (w/w) skim milk powder are added to Leuconostoc mesenteroides liquid, and are uniformly mixed, frozen, dried and ground to prepare the composition.

### Example 1: Strain Identification and Phylogenetic Tree

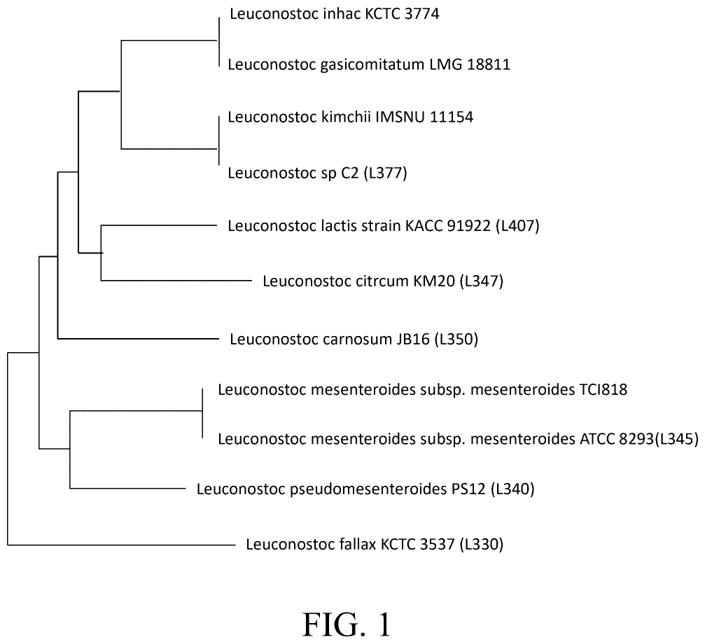

Firstly, a strain isolated from Allium tuberosum Rottler ex Spreng was subjected to strain identification. A 16S ribosomal gene (16S rDNA) sequence (SEQ ID NO: 1) of the isolated strain was obtained by polymerase chain reaction (PCR), and the total length of the gene sequence was 1,996,682. The 16S ribosomal gene (16S rDNA) sequence of a strain L337 (SEQ ID NO: 2), the 16S ribosomal gene (16S rDNA) sequence of a strain L407 (SEQ ID NO: 3), the 16S ribosomal gene (16S rDNA) sequence of a strain L347 (SEQ ID NO: 4), the 16S ribosomal gene (16S rDNA) sequence of a strain L350 (SEQ ID NO: 5), the 16S ribosomal gene (16S rDNA) sequence of a strain L345 (SEQ ID NO: 6), the 16S ribosomal gene (16S rDNA) sequence of a strain L340 (SEQ ID NO: 7), and the 16S ribosomal gene (16S rDNA) sequence of a strain L330 (SEQ ID NO: 8) for comparison were obtained in the same way. Then, each sequence above was analyzed by an analysis platform (https://www.bv-brc.org/) of the Bacterial and Viral Bioinformatics Resource Center (BV-BRC). The analysis platform carried out sequence alignment of the 16S ribosomal gene (16S rDNA) sequences of other strains of Leuconostoc (as shown in Table 1) to determine the association between a target strain and a specific taxonomic unit through the similarity scores in the alignment results. Moreover, a Neighbor-Joining (NJ) method was used for constructing a phylogenetic tree (as shown in FIG. 1). Based on this, it was found that the 16SrDNA sequence of the isolated strain was most similar to that of Leuconostoc mesenteroides subsp. Mesenteroides (accession number ATCC8293). Therefore, this isolated strain was named Leuconostoc mesenteroides subsp. mesenteroides strain TCI818.

TABLE 1

| Information of comparison strains | Genome | Similarity |
|---|---|---|
| of Leuconostoc | ID | (%) |
| Leuconostoc inhac KCTC 3774 | 927694.4 | 97.43 |
| Leuconostoc gasicomitatum LMG 18811 | 762550.4 | 97.62 |
| Leuconostoc kimchi IMSNU 11154 | 1269801500 | 97.33 |
| Leuconostoc sp C2(L377) | 979982.3 | 97.94 |
| Leuconostoc lactis strain KACC | 1246.4 | 98.08 |
| 91922 (L407) | | |
| Leuconostoc citrcum KM20 (L347) | 349519.10 | 97.83 |
| Leuconostoc carnosum JB16 (L350) | 1229758.3 | 97.36 |
| Leuconostoc mesenteroides | 203120.7 | 99.32 |
| subsp. mesenteroides ATCC 8293(L345) | | |
| Leuconostoc pseudomesenteroides | 1339247.3 | 80.42 |
| PS12 (L340) | | |
| Leuconostoc fallax KCTC 3537 (L330) | 907931.3 | 95.45 |

Here, some of the strains in Table 1 above were purchased as comparison strains and named as a strain L407, a strain L377, a strain L345, a strain L347, a strain L350, a strain L340 and a strain L330 for subsequent comparison experiments.

### Example 2: Preservation and Culture Experiments of Leuconostoc mesenteroides

The isolated Leuconostoc mesenteroides was inoculated into an MRS medium (BD Difco™ Lactobacilli MRS Broth, 1% (v/v)) at an inoculation amount of 1% (about 1×10⁴ CFU/mL), and cultured at 37° C. in an anaerobic environment for 24 h to obtain TCI818 bacterial liquid.

The TCI818 bacterial liquid was centrifuged at a rotating speed of 5,000 rpm for 20 min to separate out supernatant and Leuconostoc mesenteroides cells, and the supernatant was filtered by a 0.2 μm filter membrane to obtain filtrate that was a TCI818 sample. Herein, the filtrate obtained by the above-mentioned methods was a cell-free supernatant (namely, the TCI818 sample contained metabolites of the Leuconostoc mesenteroides).

### Example 3: Simulated Gastrointestinal Digestion Experiments

The Leuconostoc mesenteroides was tested in simulated gastric juice (pH 3) and simulated intestinal juice (pH 7) to determine the acid-base tolerance of the Leuconostoc mesenteroides TCI818 in digestive tract of an organism.

### 3-1. Preparation of Agents:

0.5% w/v of saline (PBS) was prepared, and sterilized at high temperature and high pressure.

Preparation of simulated gastric juice: a proper amount of pepsin was dissolved into 0.5% w/v of sterile saline to make the final concentration of the pepsin be 3 g/L, and the pH was regulated to be 3.0±0.2. Then the product was filtered in a sterile operating table by a 0.22 μm filter membrane to obtain sterile artificial simulated gastric juice.

Preparation of simulated intestinal juice: a proper amount of pancreatic enzyme was weighed and dissolved into 0.5% w/v of sterile saline to make the final concentration of the pancreatic enzyme be 1 g/L, 0.45% of bile salts (not added in a control group) was added, and the pH was regulated to be 8.010.2. The product was filtered in the sterile operating table by a 0.22 μm filter membrane to obtain sterile artificial simulated intestinal juice.

### 3-2. Operation Steps:

Step 1: a bacterial count test was performed on the TCI818 bacterial liquid prepared in Example 2 serving as a first sample.

Step 2:200 μL of TCI818 bacterial liquid prepared in Example 2 was added to a 1.5 ml microcentrifuge tube containing 300 μL of 0.5% w/v sterile saline and 1 ml of simulated gastric juice. After mixing evenly, the microcentrifuge tube was placed in an anaerobic incubator for culture at 37° C. for 2 h to obtain a second sample of a gastric acid simulation test, and sampling was performed for bacterial count test.

Step 3: step 2 was repeated, the centrifuge tube was centrifuged, supernatant was removed, then 1 ml of artificial intestinal juice was added, and the microcentrifuge tube was placed in the anaerobic incubator for culture at 37° C. for 2 h to obtain a third sample of an intestinal juice simulation test, and sampling was performed for bacterial count test.

Step 4: the first, second and third samples were diluted by 10-fold serial dilution, and 100 μL of diluent was coated on an MRS agar plate, which was placed at in the anaerobic incubator for culture at 37° C. for 2 d. The bacterial count in each group was counted by a plate count method after 2 d, and the content of viable bacteria per gram was back calculated.

### 3-3. Experimental Results:

The viable count of the first sample was 11.34 log CFU/mL, the viable count of the second sample was 10.88 log CFU/mL, and the viable count of the third sample was 10.23 log CFU/mL. The log CFU/mL represents colony-forming units (CFU) per milliliter of bacterial liquid, expressed in logarithm (log).

In view of above, the survival rate of Leuconostoc mesenteroides TCI818 in the simulated gastric environment (pH of 3-4) was 96% or more, and the survival rate of Leuconostoc mesenteroides TCI818 in the simulated intestinal environment (pH value 7) was 90% or more. Therefore, the Leuconostoc mesenteroides TCI818 has the function of resisting gastric acid and bile salts.

### Example 4: Lipid Droplet Accumulation Test

Fat is stored in adipocytes in a form of lipid droplets. Based on this, in this test, stained lipid droplets were analyzed to observe the number of the lipid droplets in the cells to confirm the state of fat accumulation. Subsequently, a staining agent was dissolved out and analyzed as a quantitative numerical indicator.

### 4-1. Description of Test Materials and Devices:

Cell lines: mouse bone marrow stromal cells (OP9 cells), OP9 cell from OP9 cell lines (ATCC® CRL-2749™) purchased from American Type Culture Collection.

Pre-adipocyte expansion medium: 90% Minimum Essential Medium Alpha (MEMα, Brand: Gibco), 20% Fetal Bovine Serum (Brand: Gibco) and 1% Penicillin-streptomycin (Brand: Gibco).

Differentiation medium: 86% Minimum Essential Medium Alpha (MEMα, Brand: Gibco), 10% Fetal Bovine Serum (Brand: Gibco), 1% Penicillin-streptomycin (Brand: Gibco), 0.1% Dexamethasone (Brand: Sigma-Aldrich), 1% Insulin (Brand: Sigma-Aldrich), 0.5% Indomethacin (Brand: Sigma-Aldrich) and 1.4% 3-Isobutyl-1-methylxanthine (IBMX, Brand: Sigma-Aldrich).

Agents: Oil-red O staining agent (Brand: Sigma), 10% Formaldehyde (Brand: ECHO), 100% Isopropanol (Brand ECHO), Dulbecco's phosphate buffered saline (Brand: Gibco, hereinafter referred to as PBS).

Devices: a microscope (Brand: zeiss), and a full spectrum optical analyzer (Brand: BioTek Epoch).

### 4-2. Test Process

Firstly, the OP9 cells were inoculated at 8×10⁴ cells per well into each well of a 24-well plate containing 500 μL of pre-adipocyte expansion medium, and cultured at 37° C. for 7 d. During the culture period of 7 d, 500 μL of fresh differentiation medium was replaced once every 3 d. After culturing for 7 d, the formation of lipid droplets in the cells in each well was observed with the microscope (Brand: ZEISS) to confirm that the cells were completely differentiated into adipocytes for subsequent experiments.

Experimental group: 500 μL of differentiation medium containing 0.5 μL of TCI818 samples cultivated in Example 2 (i.e., a concentration of 0.1%) added to each well was used as a test sample, and the test sample was added to a medium containing differentiated adipocytes and cultured at 37° C. for 7 d. During the cell treatment period of 7 d, the medium was replaced every 3 d.

Control group: 500 mL of differentiation medium containing 0.5 μL of MRS broth (i.e., a concentration of 0.1%) added to each well was used as a control sample, and the control sample was added to a medium containing differentiated adipocytes and cultured at 37° C. for 7 d. During the cell treatment period of 7 d, the medium was replaced every 3 d.

Blank group: no treatment was performed, namely, no other compounds were additionally added to the differentiation medium containing the differentiated adipocytes, and the culturing was performed at 37° C. for 7 d. During the cell treatment period of 7 d, the medium was replaced every 3 d.

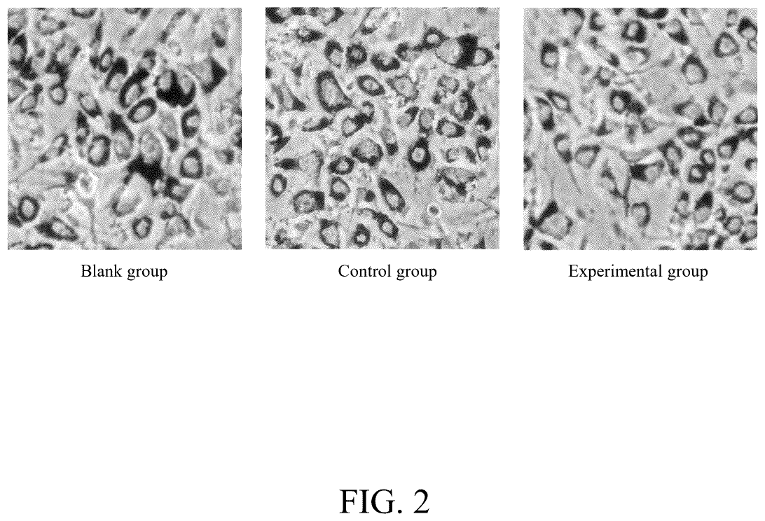

Then, staining was performed with red oil O by the following steps. After cell treatment for 7 d, the medium was removed, the adipocytes were washed twice with 1 mL of PBS, and 1 mL of 10% formaldehyde was added and reacted at room temperature for 30 min to fix the adipocytes. Next, the formaldehyde was removed, then the adipocytes were gently washed twice with 1 mL of PBS, then 1 mL of 60% isopropanol was added to cells in each well, and after reacting for 1 min, the isopropanol was removed, 1 mL of oil red O acting solution was added to react with the adipocytes at room temperature for 1 h, then the oil red O acting solution acting with the adipocytes was removed, the adipocytes were quickly destained with 1 mL of 60% isopropanol for 5 s and observed by the microscope, and the cells (the magnification of 400×) were photographed. The result is shown in FIG. 2.

Subsequently, each stained group was subjected to red oil O quantification according to the following steps. 100% of isopropanol was added to each well, and reacted for 10 min on a shaker to dissolve lipid droplets. Then, 100 μL of solution was transferred from each well to a 96-well plate, and the absorbance value (OD510 nm) of each well was read by the full spectrum optical analyzer under the wavelength of 510 nm.

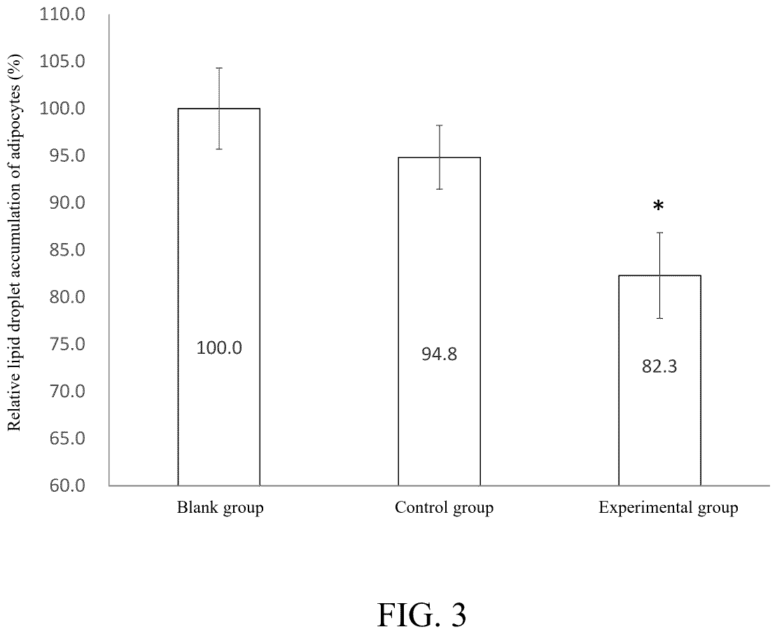

After measurement, the measured absorbance value was substituted into the following formula I to calculate the lipid droplet accumulation (%). In other words, the lipid droplet accumulation (%) of each group was calculated by taking the lipid droplet accumulation of the blank group as 100%; and the result is shown in FIG. 3.

Lipid droplet accumulation (%)=(OD₅₁₀ sample/OD₅₁₀ control)×100% (1) Formula I

OD₅₁₀ sample represents the absorbance value of the group to be converted, and OD₅₁₀ control represents the absorbance value of the blank group.

### 4-3. Experimental Results:

With reference to FIG. 2, it is observed that the number of red (dark) lipid droplets in the cells in the blank group or the control group is obviously more than that of the lipid droplets in cells in the experimental group, and the cell size is larger. Thus, the Leuconostoc mesenteroides can effectively inhibit the size of adipocytes, and then the weight losing function is achieved.

With reference to FIG. 3, when the lipid droplet accumulation of the blank group is 100%, the lipid droplet accumulation of the control group is only 94.8%, and the lipid droplet accumulation in the experimental group is remarkably reduced to be only 82.3%. Therefore, the Leuconostoc mesenteroides can effectively inhibit fat accumulation, has the function of reducing fat formation of the subject, and then achieves the function of reducing body fat.

### Example 5: Cholesterol Metabolism Test of the Liver

The liver plays a key role in cholesterol metabolism, it is responsible for synthesizing and decomposing cholesterol and converting it into bile acids for excretion. The liver also regulates the storage and release of cholesterol, affecting the level of cholesterol in the blood. In this test, NBD-Cholesterol, a fluorescent-labeled cholesterol derivative, was used for observing the effect of the Leuconostoc mesenteroides on liver cholesterol metabolism.

In the test, U18666A was used as a cholesterol binder which has the effect of inhibiting the transportation of cholesterol in cells. The inhibition effect might cause the accumulation of the cholesterol in the cells, thereby influencing the metabolic pathway of the cholesterol. Therefore, in the test, the cholesterol binder was used as a positive control group for verifying the sensitivity and the reactivity of a test system on the metabolic change of the cholesterol.

### 5-1. Description of Test Materials and Devices:

Cells: human hepatoma cells Hep G2 (hereinafter referred to as hepatocytes), from Hep G2 cell lines (ATCC HB-8065™) purchased from American Type Culture Collection (ATCC®), hereinafter referred to as hepatocytes.

Medium: 90% DMEM (Dulbecco's Modified Eagle Medium, Gibco; Cat. 11965-092), 10% Fetal Bovine Serum, and 1% penicillin-streptomycin (Gibco; Cat. 15140122).

Agents: DPBS Duchenne phosphate buffer (purchased from Gibco; Cat. 14200-075), cell Lysis Buffer and Cholesterol Binder U18666A (Sigma-Aldrich (MilliporeSigma)).

Devices: a Cholesterol Uptake Cell-based Assay Kit (Brand: Cayman) containing NBD cholesterol, and a flow cytometer (Brand: BD).

### 5-2. Test Process

Firstly, liver cells were inoculated at 4×10⁵ cells per well into each well of a 6-well plate, and cultured at 37° C. overnight for subsequent experiments.

Experimental group: 500 μL of medium containing 0.5 μL of TCI818 samples cultivated in Example 2 (i.e., a concentration of 0.1%) added to each well was used as a test sample, and the test sample was added to a serum-free medium containing 20 μg/ml of NBD cholesterol, and cultured at 37° C. for 24 h.

Positive control group: 500 μL of medium containing 0.5 μL of cholesterol binders (i.e., a concentration of 0.1%) added to each well was used as a control sample, and the control sample was added to a serum-free medium containing 20 μg/ml of NBD cholesterol, and cultured at 37° C. for 24 h.

Blank group: no treatment was performed, that is, no other compounds were additionally added to the serum-free medium containing 20 μg/ml of NBD cholesterol, and culturing was performed at 37° C. for 24 h. The absorption state of the liver cells on cholesterol under normal metabolism was simulated.

After the supernatant of the cultured liver cells in each group was removed, the liver cells were washed once with the DPBS buffer, and then the cell lysis buffer was added for 3-5 min to lyse cell membranes so as to respectively form liver cell lysates. The liver cell lysates were beaten to suspend the cells, the cells were added to the medium for neutralization, then centrifugation was performed, and the supernatant was removed after centrifugation.

Then, the cholesterol uptake cell-based assay kit was used for treatment, and a fluorescent signal was read by the flow cytometer and converted into the content of cholesterol in the liver cells.

### 5-3. Experimental Results:

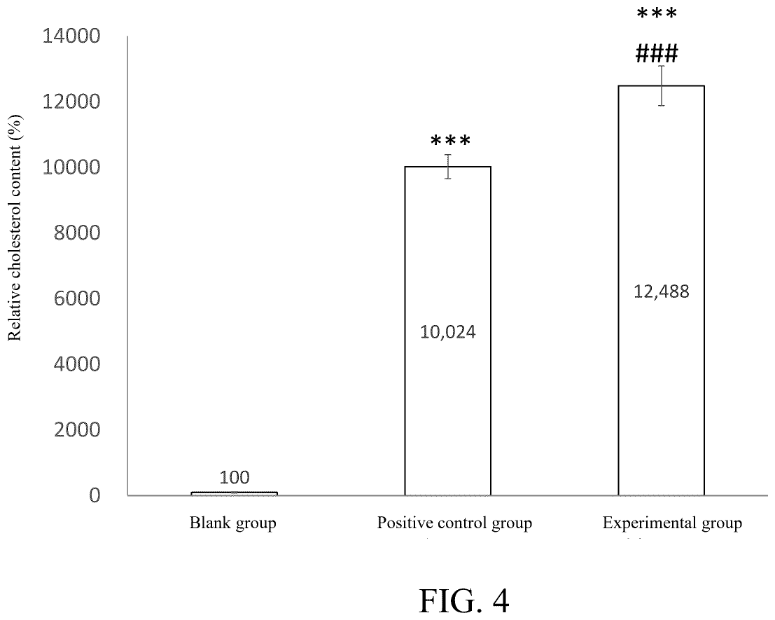

It is to be particularly noted that in FIG. 4, what is shown is presented by a relative ratio, namely, the quantitative result of the blank group was treated as 100 to convert the quantitative result of the experimental group into an expression level relative to the blank group. A GraphPad PrismOne-way ANOVA formula was used for calculation, and whether there was a statistically significant difference was analyzed. In the figure, “*” represents that the p-value relative to the blank group is less than 0.05, “**” represents that the p-value relative to the blank group is less than 0.01, “***” represents that the p-value relative to the blank group is less than 0.001, “#” represents that the p-value relative to the control group is less than 0.05, “##” represents that the p-value relative to the control group is less than 0.01, and “###” represents that the p-value relative to the control group is less than 0.001.

With reference to FIG. 4, in a case of 100% cholesterol content in the blank group, the relative cholesterol content of the positive control was 10024%.

In a case of 100% cholesterol content in the blank group, the relative cholesterol content of the experimental group was as high as 12488%, which was also higher than that of the positive control group. In other words, the Leuconostoc mesenteroides can effectively increase the metabolic rate of cholesterol, which is more efficient than known cholesterol binders, and promotes the absorption of cholesterol by the liver, thereby reducing the content of blood lipids.

### Example 6: Cholesterol-Related Gene Expression

### 6-1. Test Target and Solution Configuration

Cells: human hepatoma cells Hep G2, from Hep G2 cell lines (ATCC HB-8065™) purchased from the American Type Culture Collection (ATCC®), hereinafter referred to as hepatocytes.

Medium: 90% DMEM (Dulbecco's Modified Eagle Medium, Gibco; Cat. 11965-092), 10% Fetal Bovine Serum (Gibco), and 1% penicillin-streptomycin (Gibco; Cat. 15140122).

Agent: DPBS Duchenne phosphate buffer (purchased from Gibco; Cat. 14200-075), and cell Lysis Buffer.

Devices: RNA extraction kit (purchased from Geneaid, Taiwan, Lot No.FC24015-G), reverse transcriptase (SuperScript® III Reverse Transcriptase purchased from Invitrogene, Inc., USA, No. 18080-051), Primer set, qPCR kit (KAPA CYBR FAST qPCR Kits (2×) (KAPA Biosystems)), and ABI StepOnePlus™ Real-Time PCR system (Thermo Fisher Scientific, USA).

### 6-2. Operation Process

Firstly, 1×10⁶ liver cells and 2 mL of medium were inoculated into each well of a 6-well plate and cultured overnight. The cultured liver cells were divided into three groups: a blank group, a control group and an experimental group.

Blank group: no treatment was performed, namely, other compounds were not additionally added to the medium, the state of the cells under normal metabolism conditions was simulated, and then the cells were cultured at 37° C. for 24 h.

Control group: 500 μL of medium containing 0.5 μL of MRS broth in Example 2 (i.e., a concentration of 0.1%) added to each well was used as a test sample, and the test sample was cultured at 37° C. for 24 h. Here, whether the MRS medium had an influence on the test or not was further observed.

Experimental group: 500 μL of medium containing 0.5 μL of TCI818 samples cultivated in Example 2 (i.e., a concentration of 0.1%) added to each well was used as a test sample, and the test sample was cultured at 37° C. for 24 h.

After removing the supernatant of the cultured liver cells of each group, the liver cells were washed once with the DPBS buffer, and 600 μL of cell lysis buffer was added to lyse cell membranes to form liver cell lysates respectively.

Then, RNA in the liver cell lysate of each group was collected by the RNA extraction kit respectively. Then the RNA extracted from each group was taken out as a template, and reverse transcription was performed by virtue of reverse transcriptase through primer binding to generate corresponding cDNA. Subsequently, quantitative real-time reverse transcription polymerase chain reaction was performed on the reverse transcription products of each group with primer groups in Table 2 by the ABI StepOnePlus™ Real-Time PCR system and a qPCR kit to observe the gene expression level of the liver cells of each group.

TABLE 2

| | Name | | | Length |
|---|---|---|---|---|
| Target | of | No. of | | of |
| gene | primer | sequence | Sequence | primer |
| CETP | CETP-F | SEQ ID | GGCCAAGTCAAG | 20 |
| | | NO: 9 | TATGGGTTG | |
| | CETP-R | SEQ ID | ACAGACACGTTC | 18 |
| | | NO: 10 | TGAATGGAGA | |
| SCARB1 | SCARB1-F | SEQ ID | ACTCCGACTCTG | 20 |
| | | NO: 11 | GGCTCTTCA | |
| | SCARB1-R | SEQ ID | GGCCTCCGGGCT | 18 |
| | | NO: 12 | GTAGAA | |
| LDLR | LDLR-F | SEQ ID | TTCACTCCATCT | 22 |
| | | NO: 13 | CAAGCATCGA | |
| | LDLR-R | SEQ ID | GGACAGTAGGTT | 23 |
| | | NO: 14 | TTCAGCCAACA | |

In Table 2, F is a Forward primer and R is a Reverse primer.

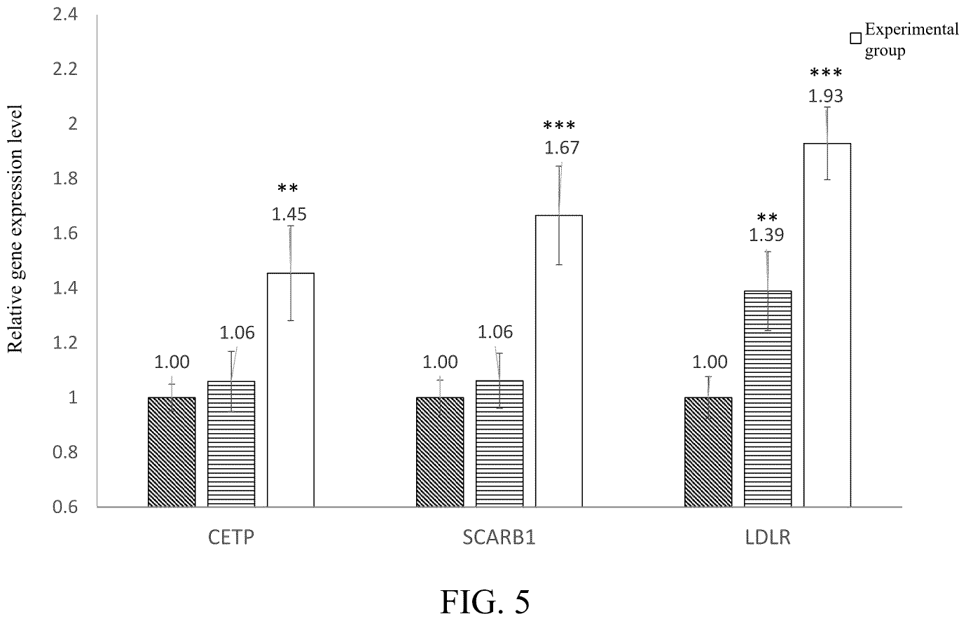

Here, the instrument settings for the quantitative real-time reverse transcription polymerase chain reaction were as follows: the reaction was performed at 95° C. for 1 s and at 60° C. for 20 s, 40 cycles in total, and relative gene quantification was performed by a 2-ΔΔCt method. Here, the mRNA expression level of each gene could be indirectly quantified by the quantitative real-time reverse transcription polymerase chain reaction using cDNA, and then the expression level of the protein encoded by each gene could be inferred, as shown in FIG. 5.

### 6-3. Discussion of Results

It is to be particularly noted that in FIG. 5, what is shown is presented by a relative ratio, namely, the quantitative result of the blank group was treated as 1 to convert the quantitative result of the experimental group into an expression level relative to the blank group. An STDEV formula of Excel software was used for calculating the standard deviation, and the one-tailed Student t-test was used in Excel software to analyze whether there was a statistically significant difference. In the figure, “*” represents that the p-value relative to the blank group is less than 0.05, “**” represents that the p-value relative to the blank group is less than 0.01, and “***” represents that the p-value relative to the blank group is less than 0.001.

With reference to FIG. 5, when the expression level of the CETP gene in the blank group is 1, the expression level of the CETP gene in the control group relative to the blank group is 1.06, and the expression level of the relative CETP gene in the experimental group is 1.45. That is, the expression level of the CETP gene in the experimental group is significantly increased compared to the blank group.

When the expression level of the SCARB1 gene in the blank group is 1, the expression level of the SCARB1 gene in the control group relative to the blank group is 1.06, and the expression level of the relative SCARB1 gene in the experimental group is 1.67. That is, the expression level of the SCARB1 gene in the experimental group is significantly increased compared to the blank group.

When the expression level of the LDLR gene in the blank group is 1, the expression level in the LDLR gene in the control group relative to the blank group is 1.39, and the expression level of the relative LDLR gene in the experimental group is 1.93. That is, the expression level of the LDLR gene in the experimental group is significantly increased compared to the blank group.

Here, the expression levels of the three genes in the experimental group were all significantly increased compared to the blank group, while the expression of only one gene in the control group was increased, and the expressions of the other two genes were not increased. Therefore, the Leuconostoc mesenteroides TCI818 can significantly increase the expression level of cholesterol-related genes, while the control group does not have such an effect.

Therefore, the liver cells can promote the production of high-density cholesterol and increase the metabolic efficiency of cholesterol with the assistance of the Leuconostoc mesenteroides, thereby reducing the content of blood lipids.

### Example 7: Incretin Secretion Promotion Test

Incretin (Glucagon-like peptide-1, GLP-1) is the glycoprotein-like peptide secreted by intestinal endocrine cells (L cells) on the small intestinal wall, and has the effects of promoting insulin secretion and inhibiting glycoprotein secretion, thereby regulating blood glucose and inhibiting appetite to achieve the effect of controlling body weight.

In this test, it was to observe the GLP-1 secretion conditions of the intestinal endocrine cells in the experimental group (with sample added), the positive control group (with glucose added) and the blank group (without addition).

### 7-1. Description of Test Materials and Devices:

Cell line: human colorectal adenocarcinoma cells, from NCL-H716 cell lines (CCL-251) purchased from American Type Culture Collection (ATCC®), hereinafter referred to as rectal cells.

### Medium:

1. RPMI-1640 medium (purchased from Gibco), with 2 mM L-Glutamine (Gibco), 10% Fetal Bovine Serum (Gibco), and 1% penicillin-streptomycin (Gibco; Cat. 15140122). 2. DMEM high glucose (Gibco). 3. DMEM without glucose (Gibco).

Agents: phosphodiesterase inhibitor (IBMX, purchased from Sigma), Forskolin (Sigma), and Dulbecco's phosphate buffered saline (Gibco, hereinafter referred to as PBS).

Devices: a full spectrum optical analyzer (Brand: BioTek Epoch), and an incretin assay agent kit (GLP-1 ELISA KIT, model: CEA804 Mi) for assay.

### 7-2. Test Process:

The rectal cells were cultured with the RPMI-1640 medium, then the cultured rectal cells were respectively inoculated to a 24-well plate (3×10⁵/well) by Matrix gel; and after the Matrix gel was solidified, the DMEM high glucose was added to culture the cells overnight.

The DMEM without glucose with 10 μm Forskolin and 10 μm IBMX was replaced and then treated for 4 h, and then the rectal cells were divided into three groups, namely, a blank group, a control group and an experimental group. Here, the Forskolin is an activator of adenylate cyclase and can increase the level of CAMP in the cells; and the IBMX is an inhibitor of phosphodiesterase and can prevent the degradation of the CAMP. The Forskolin and the IBMX are combined for use to obviously increase the concentration of the CAMP in the cells, thereby affecting the signal transduction and functions of the cells. The DMEM without glucose can avoid the interference of glucose on experimental results and ensure that the observed effects are mainly from the actions of the Forskolin and the IBMX.

Blank group: the DMEM without glucose was replaced by 500 μL/well PBS containing 1 mM CaCl₂, and 10 μm Forskolin and 10 μm IBMX were added, and stood overnight.

Positive control group: the DMEM without glucose was replaced by 500 μL/well PBS containing 1 mM CaCl₂), and 30 mM glucose, 10 μm Forskolin and 10 μm IBMX were added, and stood overnight.

Experimental group: the DMEM without glucose was replaced by 500 μL/well PBS containing 1 mM CaCl₂, the Leuconostoc mesenteroides cultured in Example 2 was added as a test sample (a concentration of 0.1%), 10 μm Forskolin and 10 μm IBMX were added, and stood overnight.

The PBS of each group was sucked, and the content of GLP-1 secreted into the culture solution by the rectal cells was detected with the incretin assay agent kit.

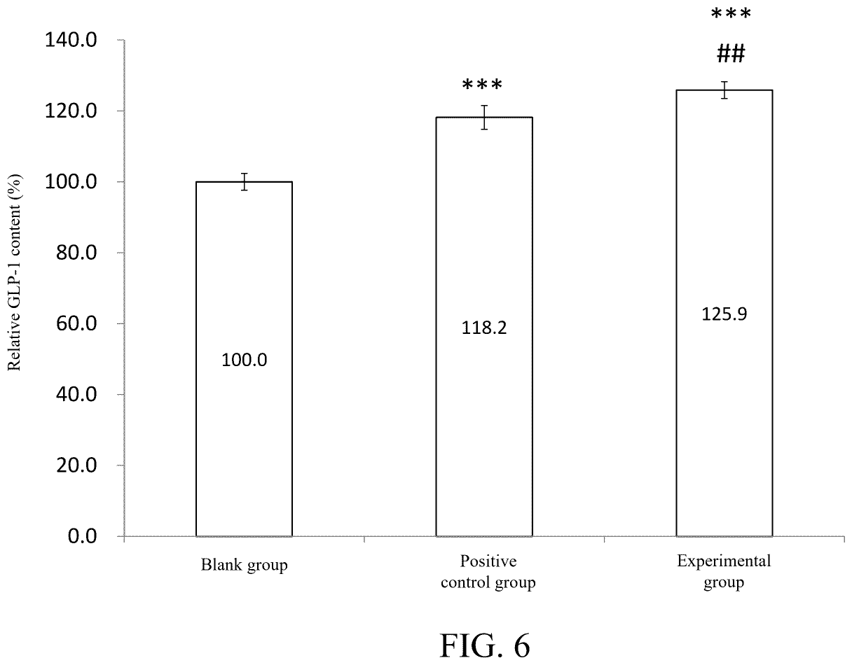

Finally, the absorbance value of each group at OD450 nm was read by the ELISA reader. The relative GLP-1 content of each group was converted by comparing and converting the absorbance value with those of the blank group (the relative GLP-1 content was treated as 100%); and the result is shown in FIG. 6.

### 7-3. Experimental Results:

The results were subjected to student t-test by the Excel software to determine whether there was a statistically significant difference between the two sample populations, as shown in FIG. 6 (in the figure, “*” represents a p-value less than 0.05, “**” represents a p-value less than 0.01, and “***” represents a p-value less than 0.001, “#” represents a p-value less than 0.05 relative to the control group, “##” represents a p-value less than 0.01 relative to the control group, and “###” represents a p-value relative to the control group was less than 0.001. The more “*” there are, the more significant the statistical difference is.

With reference to FIG. 6, compared with the blank group (normal cell metabolism), the relative GLP-1 content of the control group is 118.2%. In other words, the rectal cells will secrete more GLP-1 in a high-glucose environment, and the GLP-1 content is obviously increased by 18.2% compared with that of the blank group.

Compared with the blank group, the relative GLP-1 content of the experimental group was 125.9%. In other words, the relative GLP-1 content of the experimental group was higher than that of either the blank group or the control group. Therefore, the Leuconostoc mesenteroides TCI818 can effectively promote the secretion of GLP-1 by rectal cells. Further, it promotes insulin secretion and inhibits the secretion of glucagon, thereby regulating blood glucose and suppressing appetite to achieve the effect of controlling body weight.

### Example 8: BHB Secretion Promotion Test

β-hydroxybutyrate (hereinafter referred to as BHB) is a type of ketone, which is usually synthesized in the liver. The metabolic response deteriorates with age, the synthesis efficiency of the liver will gradually decline, and as a result, the amount of BHB synthesis will gradually become insufficient.

### 8-1. Description of Test Materials and Devices:

Cells: human hepatoma cells Hep G2, from Hep G2 cell lines (ATCC HB-8065™) purchased from the American Type Culture Collection (ATCC®), hereinafter referred to as hepatocytes.

Medium: 90% DMEM (Dulbecco's Modified Eagle Medium, Gibco; Cat. 11965-092), 10% Fetal Bovine Serum (Gibco), and 1% penicillin-streptomycin (Gibco; Cat. 15140122).

Beta Hydroxybutyrate Assay Kit (Colorimetric), model: ab83390.

### 8-2. Test Process:

Here, the TCI818 sample cultivated in Example 2 was used as the test sample, and the comparison strains L407, L377, L345, L347, L350, L340 and L330 in Table 1 were cultivated in the same way as Example 2 as the control samples, to test whether the BHB content of the liver cells could be increased after treatment with the test samples or control samples.

Firstly, a standard curve of a BHB standard sample was made.

Cells were inoculated at 1×10⁴ cells per well into a 24-well plate containing 2 ml of pretreatment medium in each well, and cultured at the temperature of 37° C. until the cells were attached.

Then, the cells were grouped. The test sample was not added to the blank group, the TCI818 sample with a concentration of 0.125% prepared in Example 2 was added to the experimental group, the L407 strain with a concentration of 0.125% prepared in Example 1 was added to a control group L407, the L377 strain with a concentration of 0.125% prepared in Example 1 was added to a control group L377, the L345 strain with a concentration of 0.125% prepared in Example 1 was added to a control group L345, the L347 strain with a concentration of 0.125% prepared in Example 1 was added to a control group L347, the L350 strain with a concentration of 0.125% prepared in Example 1 was added to a control group L350, the L340 strain with a concentration of 0.125% prepared in Example 1 was added to a control group L340, the L330 strain with a concentration of 0.125% prepared in Example 1 was added to a control group L330, and culturing was performed for 24 h to obtain a culture solution of each group.

The cultured supernatant of each group was collected, the absorbance value of the supernatant at 450 nm was measured with the spectrophotometer, and the absorbance value of the supernatant was converted into the BHB content by the standard curve.

### 8-3. Experimental Results:

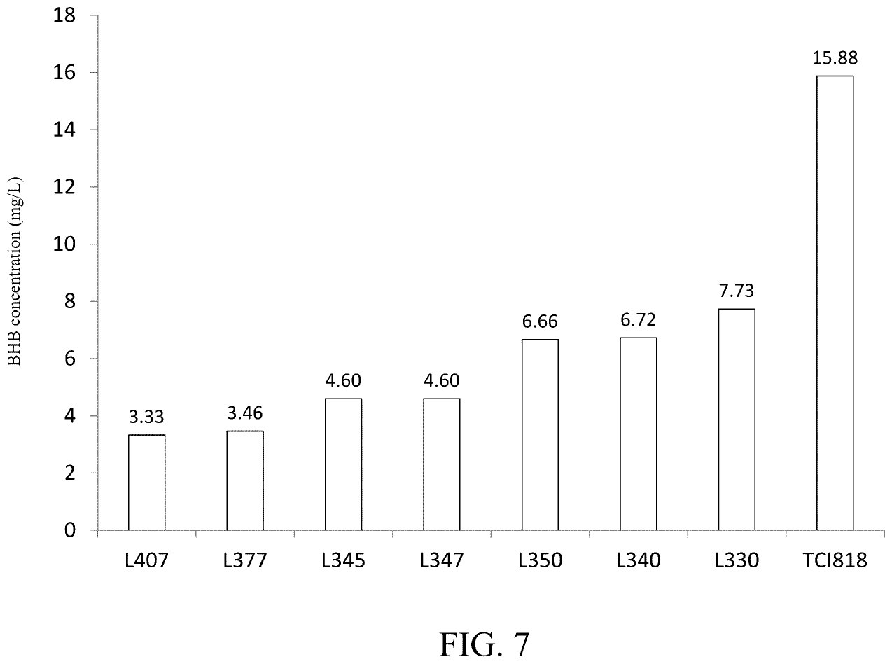

With reference to FIG. 7, the BHB secretion in the control group L407 was 3.33 mg/L, the BHB secretion in the control group L377 was 3.46 mg/L, the BHB secretion in the control group L345 was 4.60 mg/L, the BHB secretion in the control group L347 was 4.60 mg/L, the BHB secretion in the control group L350 was 6.66 mg/L, the BHB secretion in the control group L340 was 6.72 mg/L, and the BHB secretion in the control group L330 was 7.73 mg/L. On the other hand, the BHB secretion in the experimental group was 15.88 mg/L, and its effect on promoting BHB was two to five times that of other strains, and the effect was significant.

### Example 9: Human Test

9-1. Sample: a capsule prepared from the Leuconostoc mesenteroides prepared in Example 2 was used, which contained 100 mg of bacterial powder. Here, the bacterial powder was prepared by adding 2.2% (w/w) trehalose, 4.6% (w/w) lactose and 10% (w/w) skim milk powder to the Leuconostoc mesenteroides liquid prepared in Example 2, mixing evenly, freeze-drying and grinding.

9-2. Subjects: 10 subjects. The subjects were aged 20 or above and had high body fat. Here, high body fat refers to a body fat of more than 25% for men and more than 30% for women.

9-3. Test items: concentration of ketone in serum, content of ketone in urine, concentration of glycated albumin in blood, insulin resistance index (HOMA-IR), high-density lipoprotein cholesterol (HDL-C) in blood, whole body fat rate, trunk fat weight, visceral fat area, total skeletal muscle weight, and basal metabolic rate.

The concentration of ketone in serum, the concentration of glycated albumin in blood, the insulin resistance index (HOMA-IR) and the high-density lipoprotein cholesterol (HDL-C) in blood were measured by Li Zen Medical Laboratory after blood collection.

The content of ketone in urine was measured by a colorimetric method through OurHealth test paper, and the content of ketone in urine was determined according to the color of the test paper.

The whole body fat rate (%), the trunk fat weight (kg), the visceral fat area (cm²), the whole body skeletal muscle weight (kg) and the basal metabolic rate (kcal) were measured by a body composition analyzer (model: InBody 770, Biopackage Co. Ltd., Seoul, Recommonic of Korea).

### 9-4. Test Process:

The 10 subjects were administrated with 100 mg of capsule prepared from the Leuconostoc mesenteroides cells in Example 2 for four consecutive weeks. Measurement was correspondingly performed before administration (i.e. Week 0, also known as the blank group), two weeks after administration (i.e. Week 2, also known as an experimental group A) and four weeks after administration (i.e. Week 4, also known as an experimental group B).

### 9-5. Test Results:

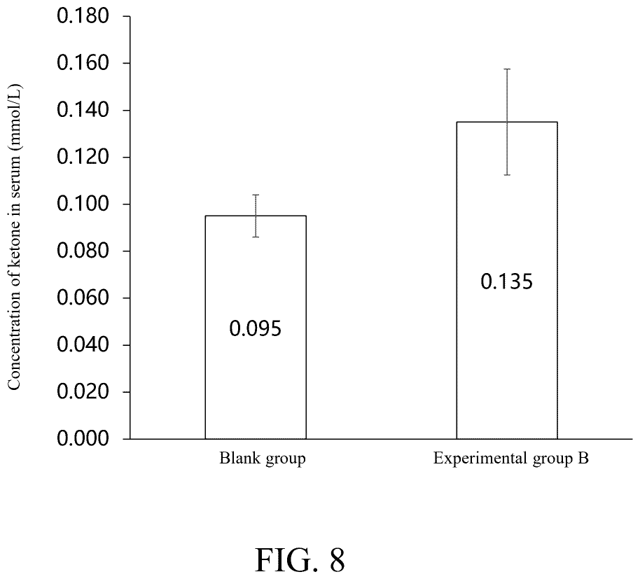

With reference to FIG. 8, after 100 mg of Leuconostoc mesenteroides was administrated every day for four weeks, the average concentration of ketone in serum of the 10 subjects was 0.095 mmol/L (blank group) and increased to 0.135 mmol/L (experimental group B) under regular diet and lifestyle. The average concentration of ketone in serum of 8 out of the 10 subjects increased, namely the proportion of subjects with improvement reached 80%, and the difference between the average concentration of ketone in serum reached 42.1% before and after administrating the Leuconostoc mesenteroides of the present invention for only four weeks. Therefore, the concentration of ketone in blood could be effectively increased by administrating 100 mg of Leuconostoc mesenteroides every day.

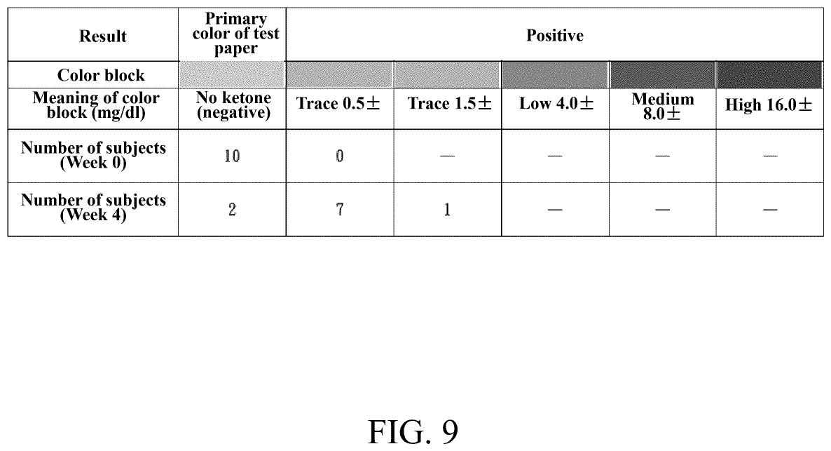

With reference to FIG. 9, none of the 10 subjects had ketones in their urine (negative) before test, and after 100 mg of Leuconostoc mesenteroides was administrated every day for four weeks, the ketone in urine of 8 out of the 10 subjects (Week 0) increased to trace (Week 4) from negative under regular diet and lifestyle, resulting in an improvement rate of 80%. Here, the trace referred to 0.5-1.5 mg/dl. This means that the content of ketone in the urine can be effectively increased by administrating 100 mg of Leuconostoc mesenteroides every day under normal diet and lifestyle.

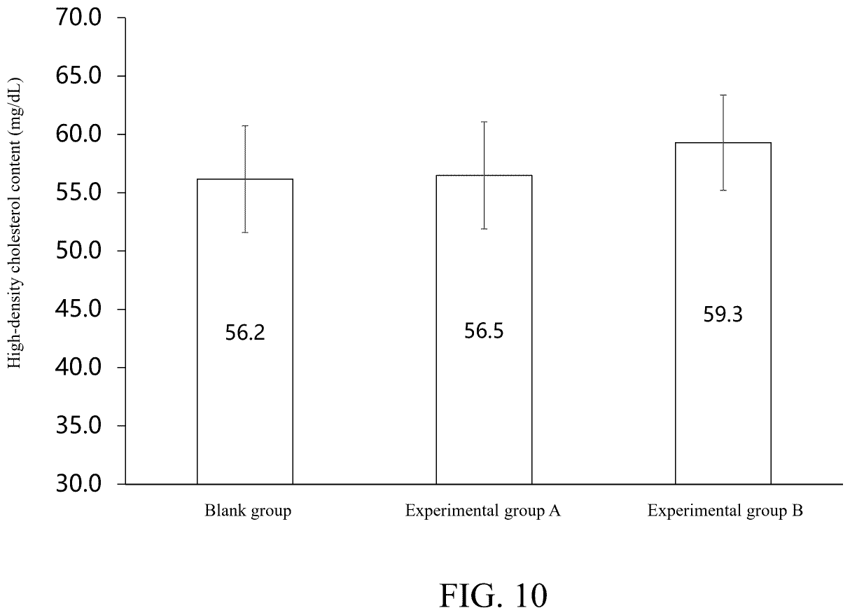

With reference to FIG. 10, the average high-density cholesterol content of the 10 subjects was 56.2 mg/dL (blank group) before test, and under regular diet and lifestyle, the average high-density cholesterol content was 56.5 mg/dL (experimental group A) after administrating 100 mg of Leuconostoc mesenteroides every day for two weeks, and the average high-density cholesterol content increased to 59.3 mg/dL (experimental group B) after administrating 100 mg of Leuconostoc mesenteroides every day for four weeks, which showed a remarkable gradual increase trend. The average high-density cholesterol content of 8 out of the 10 subjects increased, that is, the proportion of subjects with improvement reached 80%; and the average high-density cholesterol content increased by 5.5% before and after administrating the Leuconostoc mesenteroides for only four weeks. This means that the average content of high-density cholesterol in blood can be effectively increased by administrating 100 mg of Leuconostoc mesenteroides every day.

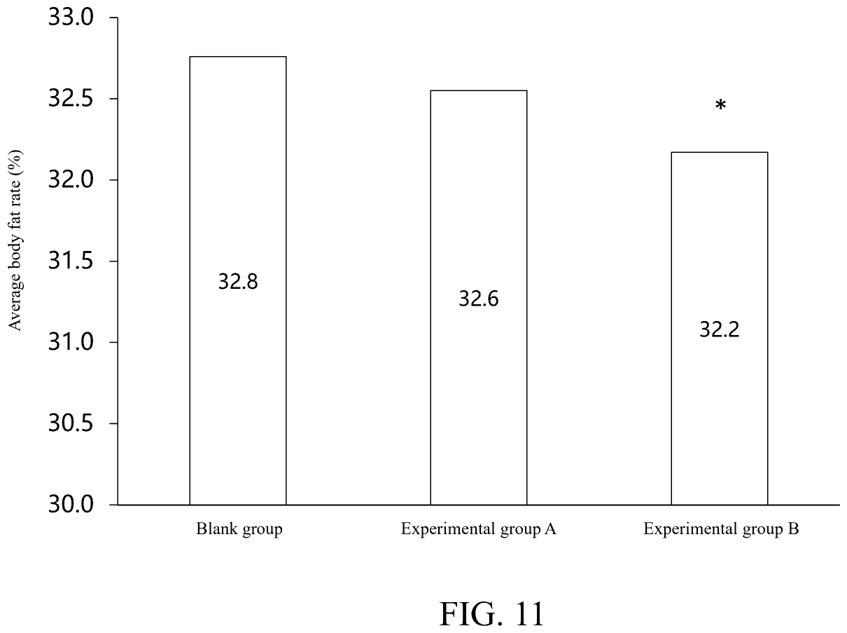

With reference to FIG. 11, the average body fat rate of the 10 subjects was 32.8% (blank group) before test, and under regular diet and lifestyle, the average body fat rate was 32.6% (experimental group A) after administrating 100 mg of Leuconostoc mesenteroides every day for two weeks, and the average body fat rate increased to 32.2% (experimental group B) after administrating 100 mg of Leuconostoc mesenteroides every day for four weeks, which showed a gradual decrease trend. The average body fat rate of 7 out of the 10 subjects decreased, that is, the proportion of subjects with improvement reached 70%, and the average body fat rate decreased by 0.6% before and after administrating the Leuconostoc mesenteroides for only four weeks. This means that the body fat can be effectively reduced by administrating 100 mg of Leuconostoc mesenteroides every day.

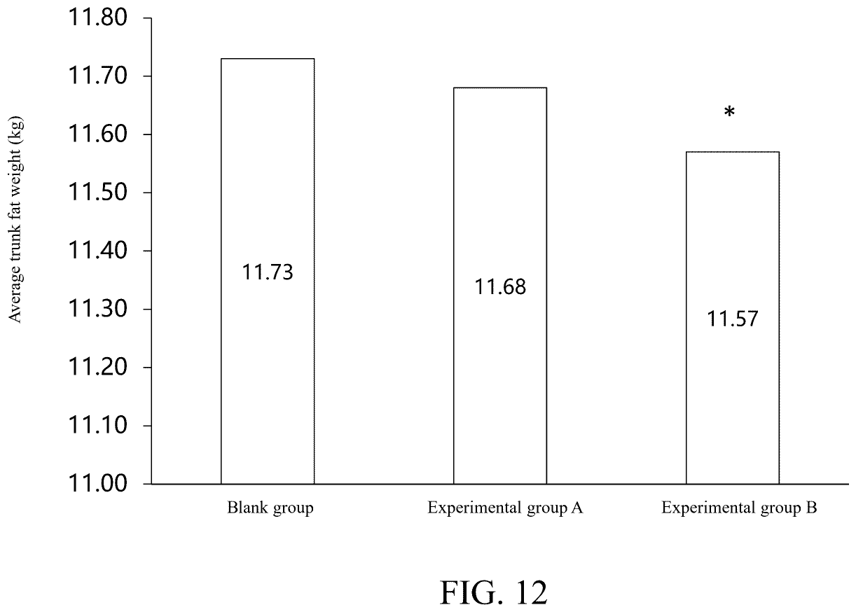

With reference to FIG. 12, the average trunk fat weight of the 10 subjects was 11.73 kg (blank group) before test, and under regular diet and lifestyle, and the average trunk fat weight was 11.68 kg (experimental group A) after administrating 100 mg of Leuconostoc mesenteroides every day for two weeks, and the average trunk fat weight decreased to 11.57 kg (experimental group B) after administrating 100 mg of Leuconostoc mesenteroides every day for four weeks, which showed a gradual decrease trend. The average trunk fat weight of 7 of the 10 subjects decreased, that is, the proportion of subjects with improvement reached 70%, and the average trunk fat weight decreased by 0.16 kg before and after administrating the Leuconostoc mesenteroides for only four weeks. This means that the trunk fat weight can be effectively reduced by administrating 100 mg of Leuconostoc mesenteroides every day.

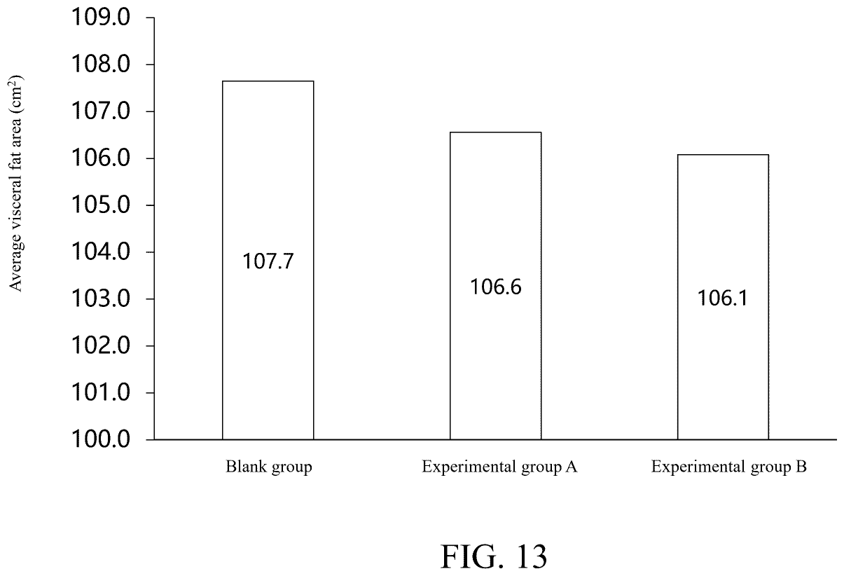

With reference to FIG. 13, the average visceral fat area of the 10 subjects was 107.7 cm² (blank group) before test, and under regular diet and lifestyle, the average visceral fat area was 106.6 cm² (experimental group A) after administrating 100 mg of Leuconostoc mesenteroides every day for two weeks, and the average visceral fat area decreased to 106.1 cm² (experimental group B) after administrating 100 mg of Leuconostoc mesenteroides every day for four weeks, which showed a gradual decrease trend. The average visceral fat area of 8 out of the 10 subjects decreased, namely, the proportion of subjects with improvement reached 80%, and the average visceral fat area decreased by 1.6 cm² before and after administrating the Leuconostoc mesenteroides for only four weeks. This means that the visceral fat area can be effectively reduced by administrating 100 mg of Leuconostoc mesenteroides every day.

According to current medical standards, the normal range for the visceral fat area is 50-100 cm² for men and 40-80 cm² for women. The average visceral fat area of 3 out of the 10 subjects exceeded the standard, and the average visceral fat area of the 3 subjects was as high as 146.9 cm² before test, and under regular diet and lifestyle, the average visceral fat area of the 3 subjects was significantly improved after administrating 100 mg of Leuconostoc mesenteroides every day for only two weeks, and the average visceral fat area decreased to 142.6 cm², not only the proportion of subjects with improvement reached 100%, but the actual decrease of visceral fat area was as high as 4.3 cm².

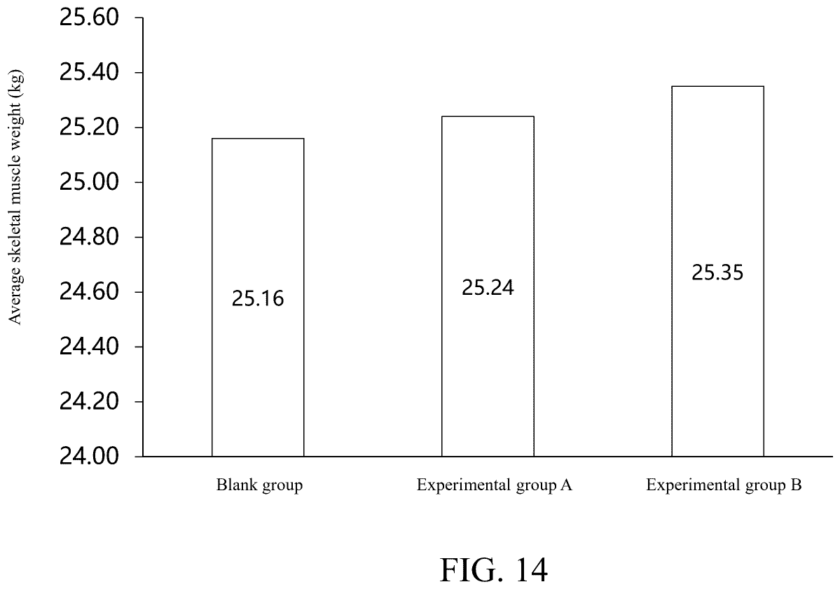

With reference to FIG. 14, the average skeletal muscle weight of the 10 subjects was 25.16 kg (blank group) before test, and under regular diet and lifestyle, the average skeletal muscle weight was 25.24 kg (experimental group A) after administrating 100 mg of Leuconostoc mesenteroides every day for two weeks, and the average skeletal muscle weight increased to 25.35 kg (experimental group B) after administrating 100 mg of Leuconostoc mesenteroides every day for four weeks, which showed a gradual increase trend. The average skeletal muscle weight of 7 out of the 10 subjects increased, that is, the proportion of subjects with improvement reached 70%, and the average skeletal muscle weight increased by 0.19 kg before and after administrating Leuconostoc mesenteroides for only four weeks. This means that the human muscle mass can be effectively increased by administrating 100 mg of Leuconostoc mesenteroides every day.

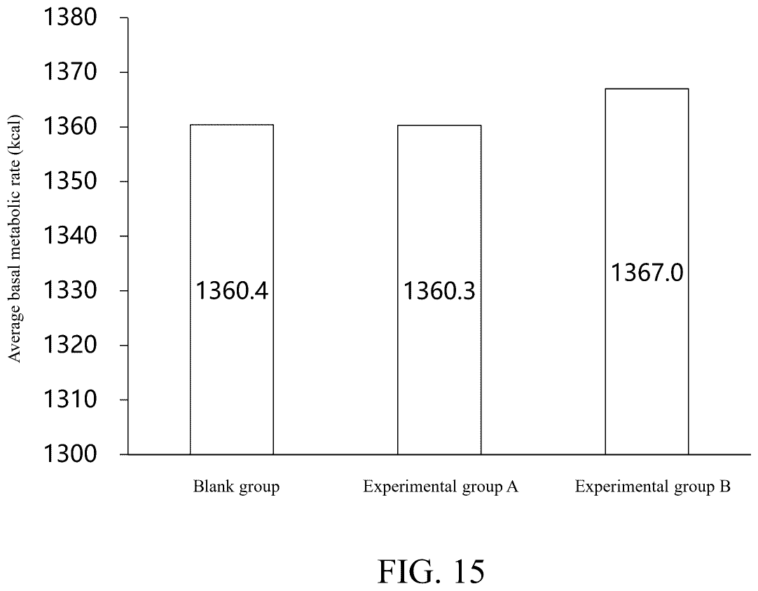

With reference to FIG. 15, the average basal metabolic rate of the 10 subjects was 1360.4 kcal (blank group) before test, and under regular diet and lifestyle, the average basal metabolic rate was 1360.3 kcal (experimental group A) after administrating 100 mg of Leuconostoc mesenteroides every day for two weeks, and the average basal metabolic rate rapidly increased to 1367.0 kcal (experimental group B) after administrating 100 mg of Leuconostoc mesenteroides every day for four weeks. The average basal metabolic rate of 6 out of the 10 subjects increased, that is, the proportion of subjects with improvement reached 60%, and the average basal metabolic rate increased by 6.6 kcal before and after administrating the Leuconostoc mesenteroides for only four weeks. This means that the basal metabolic rate of the human body can be effectively increased by administrating 100 mg of Leuconostoc mesenteroides every day.

In conclusion, the Leuconostoc mesenteroides and/or supernatant thereof in any embodiment above can promote fat metabolism, reduce lipid droplet accumulation of adipocytes, promote cholesterol metabolism, increase the content of high-density cholesterol in blood, promote production of high-density cholesterol and promote the increase of expression level of CETP gene, SCARB1 gene or LDLR gene, thus achieving the use of reducing body fat. The Leuconostoc mesenteroides and/or supernatant thereof in any embodiment can promote secretion of incretin and/or promote secretion of GLP-1 by intestinal cells, thus achieving the use of reducing appetite. The Leuconostoc mesenteroides and/or supernatant thereof in any embodiment can produce ketone, β-hydroxybutyrate, increase the concentration of ketone in serum and increase the content of ketone in urine, thus achieving the use of promoting ketone production. The Leuconostoc mesenteroides and/or supernatant thereof in any embodiment can reduce the insulin resistance index, promote secretion of incretin and/or promote secretion of GLP-1 by intestinal cells and reduce the concentration of glycated albumin, thus achieving the use of regulating fasting blood glucose.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A method for reducing body fat, comprising: administering to a subject in need thereof a composition containing an effective dose of Leuconostoc mesenteroides and/or supernatant thereof, wherein the Leuconostoc mesenteroides is Leuconostoc mesenteroides subsp. Mesenteroides deposited under accession number BCRC911146 or DSM34443.

2. The method according to claim 1, wherein the Leuconostoc mesenteroides is used for promoting fat metabolism.

3. The method according to claim 2, wherein the Leuconostoc mesenteroides is used for reducing lipid droplet accumulation in adipocytes.

4. The method according to claim 1, wherein the Leuconostoc mesenteroides is used for promoting cholesterol metabolism.

5. The method according to claim 4, wherein the Leuconostoc mesenteroides is used for increasing a content of high-density cholesterol in blood.

6. The method according to claim 4, wherein the Leuconostoc mesenteroides is used for promoting production of high-density cholesterol.

7. The method according to claim 5, wherein the Leuconostoc mesenteroides is used for promoting reduction in expression levels of CETP gene, SCARB1 gene or LDLR gene.

8. A method for reducing appetite, comprising: administering to a subject in need thereof an effective dose of Leuconostoc mesenteroides and/or supernatant thereof, wherein the Leuconostoc mesenteroides is Leuconostoc mesenteroides subsp. Mesenteroides under accession number BCRC911146 or DSM34443.

9. The method according to claim 8, wherein the Leuconostoc mesenteroides is used for promoting secretion of incretin.

10. The method according to claim 9, wherein the Leuconostoc mesenteroides is used for promoting secretion of GLP-1 by intestinal cells.

11. A method for promoting ketone production, comprising: administering to a subject in need thereof an effective dose of Leuconostoc mesenteroides and/or supernatant thereof, wherein the Leuconostoc mesenteroides is Leuconostoc mesenteroides subsp. Mesenteroides under accession number BCRC911146 or DSM34443.

12. The method according to claim 11, wherein the Leuconostoc mesenteroides is used for producing ketone.

13. The method according to claim 12, wherein the Leuconostoc mesenteroides is used for producing β-hydroxybutyrate.

14. The method according to claim 11, wherein the Leuconostoc mesenteroides is used for increasing a concentration of ketone in serum.

15. The method according to claim 11, wherein the Leuconostoc mesenteroides is used for increasing a content of ketone in urine.

16. A method for regulating fasting blood glucose, comprising: administering to a subject in need thereof with an effective dose of Leuconostoc mesenteroides and/or supernatant thereof, wherein the Leuconostoc mesenteroides is Leuconostoc mesenteroides subsp. Mesenteroides under accession number BCRC911146 or DSM34443.

17. The method according to claim 16, wherein the Leuconostoc mesenteroides is used for reducing an insulin resistance index.

18. The method according to claim 16, wherein the Leuconostoc mesenteroides is used for promoting secretion of incretin.

19. The method according to claim 18, wherein the Leuconostoc mesenteroides is used for promoting secretion of GLP-1 by intestinal cells.

20. The method according to claim 16, wherein the Leuconostoc mesenteroides is used for reducing a concentration of glycated albumin.
